(19) United States
(12) Patent Application Publication
(10) Pub. No.: US 2016/0232427 A1
(43) Pub. Date: Aug. 11, 2016

(54) SPECTRAL FRACTIONATION DETECTION OF GOLD NANOROD CONTRAST AGENTS USING OPTICAL COHERENCE TOMOGRAPHY

(71) Applicants: David HUANG, PORTLAND, OR (US); Yali JIA, PORTLAND, OR (US); Ashwath JAYAGOPAL, NASHVILLE, TN (US); Gangjun LIU, PORTLAND, OR (US)

(72) Inventors: David HUANG, PORTLAND, OR (US); Yali JIA, PORTLAND, OR (US); Ashwath JAYAGOPAL, NASHVILLE, TN (US); Gangjun LIU, PORTLAND, OR (US)

(73) Assignees: VANDERBILT UNIVERSITY, NASHVILLE, TN (US); OREGON HEALTH & SCIENCE UNIVERSITY, PORTLAND, OR (US)

(21) Appl. No.: 14/930,977

(22) Filed: Nov. 3, 2015

Related U.S. Application Data
(60) Provisional application No. 62/075,025, filed on Nov. 4, 2014.

(51) Int. Cl. G06K 9/62; G01N 33/483; G06T 5/40

(52) U.S. Cl. CPC G06K 9/6212; G06T 5/40; G01N 33/4833; G06T 2207/30024; G06T 2207/10101

(57) ABSTRACT

Methods and systems for detecting a gold nanorod (GNR) contrast agent in an optical coherence tomography (OCT) image of a sample are disclosed. In one example approach, a method comprises separating the OCT image at the location into short and long wavelength halves around a center wavelength of the OCT system, calculating a ratio between the short and long wavelength halves, and indicating a gold nanorod contrast agent at the location based on the ratio. In some examples, spectral fractionation may be employed to further divide the short and long wavelength halves into sub-bands to increase spectral contrast, reduce noise, and increase accuracy in detecting GNR in a sample.

### ACKNOWLEDGMENT OF GOVERNMENT SUPPORT

This invention was made with United States government support under the terms of grant numbers R01EY023285, R01EY024544, DK104397, and TR000128 awarded by the National Institutes of Health. The United States government has certain rights in this invention.

### FIELD

The present disclosure relates to the field of optical coherence tomography (OCT), and, more specifically, to systems and methods for detecting contrast agents using OCT.

### BACKGROUND

Optical coherence tomography (OCT) is a noninvasive and nondestructive imaging modality that is capable of providing micron-scale axial resolution for in vitro and in vivo imaging applications through the use of low-coherence interferometry (see Huang et al, Science 254, 1178-1181, (1991), which is incorporated by reference herein). OCT has been successfully integrated into pre-clinical and clinical research in the fields of ophthalmology, dermatology, cardiology, otolaryngology, and oncology, among others (see Drexler & Fujimoto, eds., Optical coherence tomography: Technology and applications, Springer Verlag, Berlin, Heidelberg, N.Y. (2008), which is incorporated by reference herein). OCT primarily relies on variations in optical scattering and absorption between tissue layers and cell types. As an example, retinal nerve fibers and pigment epithelium are more reflective than their surrounding tissue, and this type of endogenous tissue contrast is sufficient to delineate nearly all retinal sublayers that are identifiable in histology (see Drexler, J Biomed Opt 9, 47-74 (2004), which is incorporated by reference herein). OCT lacks an effective and practical contrast agent capable of cellular and molecular labeling. OCT cannot utilize typical fluorescent labeling because the fluorescence absorption and emission process destroys the coherence required for OCT (Boppart et al, J Biomed Opt 10, 41208 (2005), which is incorporated by reference herein). As a result, the search for and development of contrast agents for implementation with OCT is of significant interest.

Several extrinsic contrast mechanisms have been proposed for OCT. One approach has been to use various highly scattering agents as signal enhancers (see Lee et al, Opt Lett 28, 1546-1548 (2003), Zagaynova et al, Phys Med Biol 53, 4995-5009 (2008), and Shim et al, J Am Chem Soc 132, 8316-8324 (2010), all of which are hereby incorporated by reference). A related approach has focused on using agents that are strongly absorbing at the OCT operating wavelengths, such as indocyanine green or nanoparticles (see Yaqoob et al, J Biomed Opt 11, 054017 (2006), Au et al, Adv Mater 23, 5792-5795 (2011), and Troutman et al, Opt Lett 32, 1438-1440 (2007), all of which are incorporated by reference herein).

Within these approaches, silver or gold nanoparticles which exhibit a property known as surface plasmon resonance (SPR) have been investigated as contrast agents. These agents take advantage of SPR to overcome the extreme size-dependent reduction in the optical response seen with nanoparticles, while still retaining the preferable size for cellular interactions. Of particular interest are rod-shaped gold nanoparticles (GNR) which exhibit SPR in the near-infrared wavelengths and can be coated with polyethylene glycol to reduce cellular toxicity and functionalized to improve cellular uptake (see Oldenburg et al, Opt Express 14, 6724-6738 (2006), Akiyama et al, J Control Release 139, 81-84 (2009), Gong et al, Beilstein J Nanotechnol 5, 546-553 (2014), Alkilany et al, Small 8, 1270-1278 (2012), Krpetic et al, ACS Nano 5, 5195-5201 (2011), Huff et al, Langmuir 23, 1596-1599 (2007), and Alkilany et al, Bioconjug Chem 25, 1162-1171 (2014), all of which are incorporated by reference herein).

Previous approaches have employed contrast agents as signal enhancers or reducers. In the case of the former, because tissue reflectance usually spans a wide dynamic range due to variable incidence angle, speckle, and composition, reflectance itself may not provide sufficient contrast. In the case of strongly absorbing agents, OCT is used to detect the resulting “shadow” cast on subjacent tissue. One issue with such an approach is that the detectable shadow may complicate the determination of axial location of the labeled cell or molecule. Furthermore, there may be confounding sources of shadows, one example being the presence of blood vessels in the tissue.

A related but alternative approach utilizes magnetic particles. Such approaches take advantage of the synchronized reorientation of the particles in the presence of an oscillating magnetic field to generate contrast (see Oldenburg et al, Opt Lett 30, 747-749 (2005), which is hereby incorporated by reference). However, in such approaches, the need for synchronization of the OCT system to an alternating external magnetic field generator complicates system design. Furthermore, such approaches may require the sample to be placed into a magnet and thus may be restricted to animals and tissue that could fit into the magnet. Further still, in such approaches, all experimental apparatuses must be compatible with operation in a strong alternating magnetic field thus may be cumbersome to implement.

The use of GNR has been previously described, but each approach has limitations. Visualization of GNR particles in the anterior chamber of the eye has been demonstrated (see de la Zerda et al, Clin Experiment Ophthalmol 43, 358-366 (2015), which is hereby incorporated by reference). However, this approach relied only on the high reflectance of GNR particles relative to the aqueous fluid in the anterior chamber, which is normally clear. This approach cannot be generalized to most tissue, which also has high reflectance components that cannot be distinguished from the high reflectance of GNR.

Photothermal GNR OCT uses a modulated laser to heat up the GNR causing a periodic phase shift in the OCT signal due to thermal expansion near the absorber (see Tucker-Schwartz et al, Biomed Opt Express 3, 2881-2895 (2012), which is incorporated by reference herein). There are several disadvantages to the photothermal approach. First, a relatively high powered laser is needed to heat tissue. Such a laser would not be safe to use in some tissues, such as the eye. Second, the detection of photothermal phase shift requires the OCT beam to dwell on each position over many axial scans, making image acquisition very slow. Third, thermal diffusion limits the resolution of GNR position. Fourth, laser heating dosimetry is a complex function of depth, scattering, and absorption, making image interpretation complex.

Polarization-sensitive OCT had been used to discriminate cells and GNR by detecting the cross-polarized signal reflected from GNR (see Oldenburg et al, Opt Lett 38, 2923-2926 (2013), which is incorporated by reference herein). However, many tissue components also reflect cross-polarized light due to either birefringence (e.g. nerve fibers and collagen fibers) or depolarization (melanin particles). Olderburg et al, supra further detected motion of GNR to distinguish cells. However, in living tissue blood flow also produces motion. Therefore these approaches are impractical in living tissue.

In another approach, a GNR detection methodology was reported employing a dual wavelength-band SSOCT system (see Kim et al, Opt Lett 39, 3082-3085 (2014), which is incorporated by reference herein). In such an approach, a contrast was derived from the difference between the signals from two light sources of different wavelengths (1040 nm versus 1300 nm wavelength). Such an approach relies on a special dual-wavelength swept-source OCT system which is not commonly available and is costly to implement. Clearly new approaches to detect contrast agents using OCT are necessary.

### SUMMARY

The present disclosure is directed to methods and systems used in detecting a gold nanorod (GNR) contrast agent in an optical coherence tomography (OCT) image of a sample by detecting a spectral shift of the backscattered light from the nanorods through comparison of a ratio between short and long wavelength halves of the OCT signal. In some examples, spectral fractionation may be employed to further divide the short and long wavelength halves into sub-bands to increase spectral contrast, reduce noise, and increase accuracy in detecting GNR in a sample.

Embodiments described herein utilize GNRs specifically engineered to have a unique spectrally-encoded backscatter/reflectance by tuning their SPR peak to a wavelength that is shifted to one side of the OCT spectral band. The spectrally shifted GNR backscatter/reflection can then then be detected by a spectroscopic analysis approach referred to herein as “spectral fractionation,” and is based on a calculated ratio between short and long wavelength halves identified in an OCT image. Embodiments described herein may be used to detect the spectral signature of GNR reflectance using standard Fourier-domain OCT systems that employ only a single light source with a continuous spectrum.

Such an approach may be readily implemented on conventional Fourier-domain OCT systems without relying on specialized OCT systems to accurately detect GNR presence and GNR location in a sample imaged with OCT. Further, by averaging sub-band B-scans with different speckle patterns such an approach may lead to a reduction in speckle noise.

Embodiments herein may be advantageously used to detect cellular and molecular labeling using GNR and OCT, e.g., by utilizing GNR coated with PEG and Tat peptides. OCT with cellular and molecular labeling with GNR contrast agent has deeper penetration that traditional optical imaging of fluorescent labels and has greater spatial resolution than contrast imaging with MRI, CT, and PET, for example. Such an approach has many potential applications. For example, embodiments described herein may be used in OCT imaging to detect inflammatory cells, which play a part in many chronic diseases from uveitis (inflammation in the eye) to sudden cardiac death caused by rupture of inflamed (vulnerable) atheromatous plaque in the coronary artery. Such an approach could also be used to label and image cancer cells and assess the effectiveness of stem cell therapy in a wide variety of diseases, for example.

This Summary is provided to introduce a selection of concepts in a simplified form that are further described below in the Detailed Description. This Summary is not intended to identify key features or essential features of the disclosed subject matter, nor is it intended to be used to limit the scope of the disclosed subject matter. Furthermore, the disclosed subject matter is not limited to implementations that solve any or all disadvantages noted in any part of this disclosure.

### BRIEF DESCRIPTION OF THE SEVERAL VIEWS OF THE DRAWINGS

FIG. 1 schematically shows an example system for detecting a gold nanorod contrast agent in an OCT image of a sample in accordance with the disclosure.

FIGS. 2 and 3 schematically show example OCT systems in accordance with the disclosure.

FIG. 4 shows an example method for detecting a gold nanorod (GNR) contrast agent at a location in an optical coherence tomography (OCT) image of a sample in accordance with the disclosure.

FIG. 5 shows an example normalized extinction spectra of a GNR with a surface plasmon resonance (SPR) peak at 900 nm and a normalized intensity spectra of a spectral OCT system having a center wavelength of 840 nm.

FIG. 6 shows an example normalized extinction spectra of a GNR with a SPR peak at 980 nm and a normalized intensity spectra of a swept-source OCT system having a center wavelength of 1050 nm.

FIG. 7 shows a transmission electron micrograph of monodisperse GNR coated with polyethylene glycol (PEG) and Tat cell internalization peptides.

FIG. 8 shows graphs illustrating an example method for detecting a gold nanorod contrast agent at a location in an OCT image of a sample in accordance with the disclosure.

FIG. 9 shows pseudocolored OCT images of an intralipid sample, GNR with an SPR peak at 900 nm, and a GNR-in-intralipid sample.

FIG. 10 shows histogram distribution plots of a spectral shift of an OCT signal from a tissue phantom and GNR with an SPR peak at 980 nm.

FIG. 11 shows pseudocolored OCT images of an intralipid sample and GNR with an SPR peak at 980 nm.

FIG. 12 shows pseudocolored OCT images of a gelatin sample, unlabeled cultured retinal pigment epithelial (RPE) cells, and RPE cells labeled with GNR.

FIG. 13 schematically shows an example computing system in accordance with the disclosure.

### DETAILED DESCRIPTION

The following detailed description is directed methods and systems for detecting a gold nanorod (GNR) contrast agent at a location in an optical coherence tomography (OCT) image of a sample. In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents. Various operations may be described as multiple discrete operations in turn, in a manner that may be helpful in understanding embodiments; however, the order of description should not be construed to imply that these operations are order dependent.

As remarked above, previous OCT imaging approaches have not been able to take advantage of contrast agents capable of cellular and molecular labeling. Embodiments described herein utilize specifically engineered GNRs as contrast agents which may be used to label cells or molecules in a sample or in vivo. Embodiments of the systems and methods described herein may be used to detect GNR contrast agents in an OCT image of a sample by detecting a spectral shift of the backscattered light from the nanorods through comparison of a ratio between short and long wavelength halves of the OCT signal intensity. In some examples, spectral fractionation may be employed to further divide the short and long wavelength halves into sub-bands to increase spectral contrast, reduce noise, and increase accuracy in detecting GNR in a sample. The embodiments described herein may be employed to extend the high-resolution 3D volumetric imaging capability of OCT to include a wider variety of biological applications, for example.

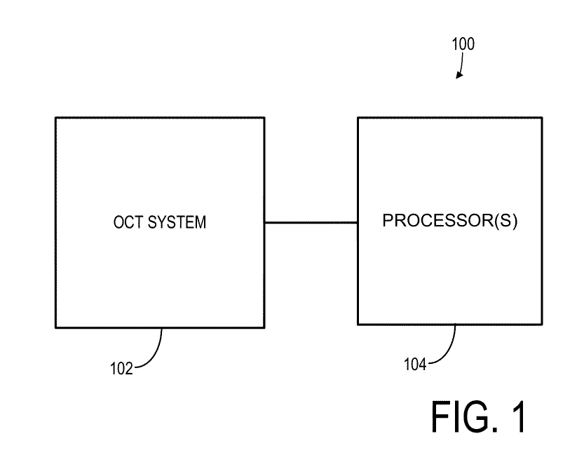

FIG. 1 schematically shows an example system 100 for detecting a GNR contrast agent in an OCT image of a sample. System 100 comprises an OCT system 102 configured to acquire an OCT image of a sample and one or more processors or computing systems 104 which are configured to implement the various processing routines described herein.

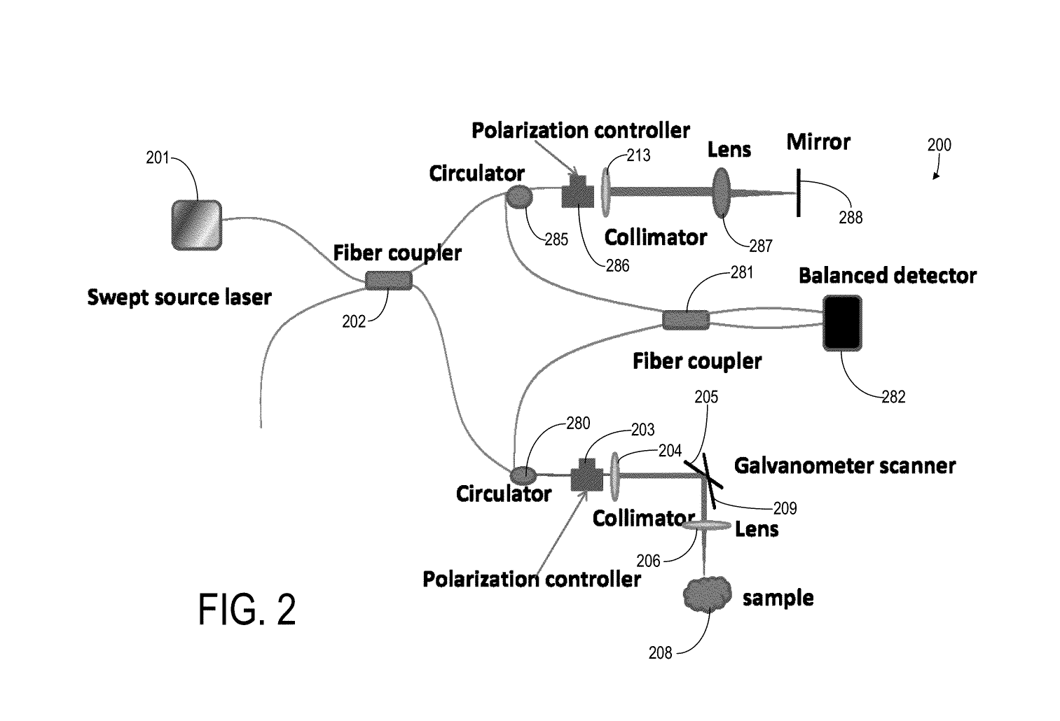

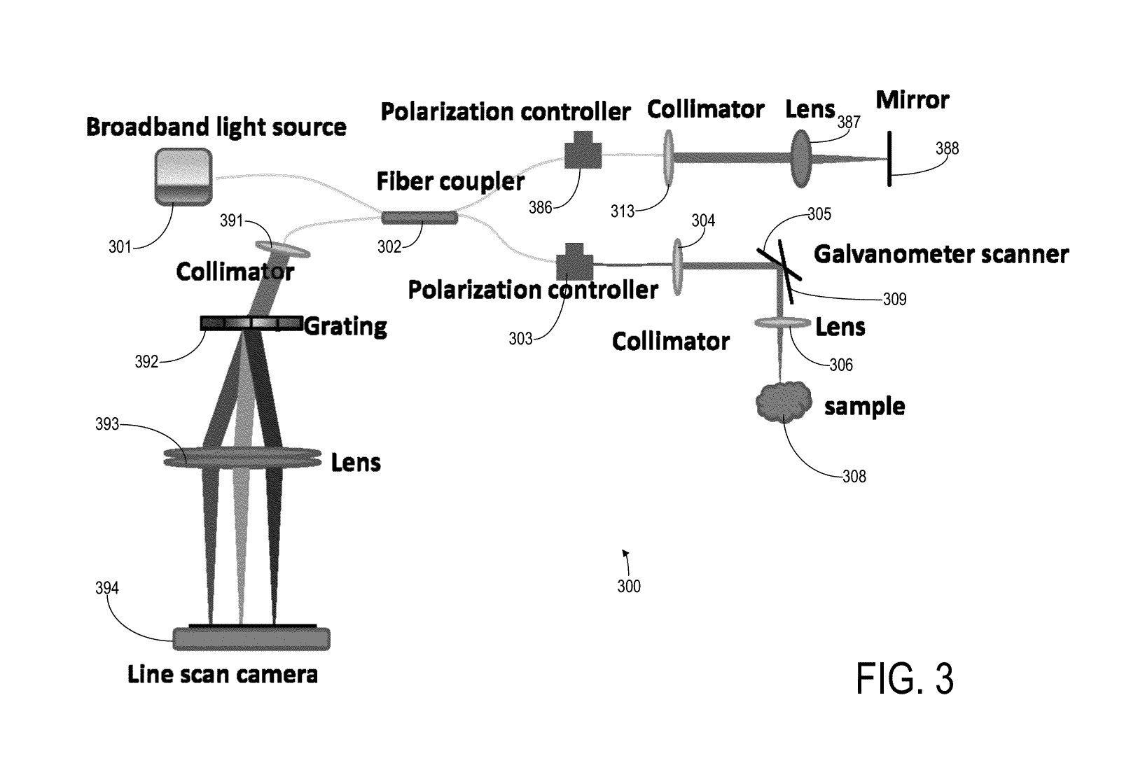

OCT system 100 may comprise any suitable Fourier-domain OCT system. In embodiments, the OCT system may have only a single light source with a continuous spectrum. Additionally, in embodiments, the OCT system may have a single center wavelength associated with the OCT system. In Fourier-domain OCT systems, the reference mirror is kept stationary and the interference between the sample and reference reflections are captured as spectral interferograms, which may be processed by inverse Fourier-transform to obtain cross-sectional images. As one example, OCT system 100 may comprise a swept-source OCT system, e.g., as shown schematically in FIG. 2 described below. In a swept-source OCT implementation of Fourier-domain OCT, the light source is a laser that is very rapidly and repetitively tuned across a wide spectrum and the spectral interferogram is captured sequentially. As another example, OCT system 100 may comprise a spectral Fourier-domain OCT system, e.g., as shown schematically in FIG. 3 described below. In the spectral OCT implementation of Fourier-domain OCT, a broad band light source is used and the spectral interferogram is captured by a grating or prism-based spectrometer. The spectrometer uses a line camera to detect the spectral interferogram in a simultaneous manner.

In various embodiments, an OCT system may be adapted to allow an operator to perform various tasks. For example, an OCT system may be adapted to allow an operator to configure and/or launch various ones of the herein described methods. In some embodiments, an OCT system may be adapted to generate, or cause to be generated, reports of various information including, for example, reports of the results of scans run on a sample.

In embodiments of OCT systems comprising a display device, data and/or other information may be displayed for an operator. In embodiments, a display device may be adapted to receive an input (e.g., by a touch screen, actuation of an icon, manipulation of an input device such as a joystick or knob, etc.) and the input may, in some cases, be communicated (actively and/or passively) to one or more processors. In various embodiments, data and/or information may be displayed, and an operator may input information in response thereto.

FIG. 2 schematically illustrates an example swept-source Fourier-domain OCT system 200 for collecting OCT image information. For example, a high-speed swept-source OCT system as described in Potsaid et al., Opt. Express 18(19), 20029-20048 (2010), which is hereby incorporated by reference, can used to implement the herein described methods. Swept-source OCT system 200 comprises a tunable laser 201. For example, tunable laser 201 (e.g., a tunable laser from Axsun Technologies, Inc, Billerica, Mass., USA) may have a wavelength of 1050 nm with 100 nm tuning range, a tuning cycle with a repetition rate of 100 kHz and a duty cycle of 50%. As another example, the tunable laser 201 may have a wavelength of 1310 nm with a 100 nm tuning range, a tuning cycle with a repetition rate of 50 kHz and a duty cycle of 50%. Light from swept source 201 can be coupled into a two by two fiber coupler 202 through a single mode optical fiber. One portion of the light (e.g., 90%) can proceed to the sample arm and the other portion of the light (e.g., 10%) can proceed to the reference arm.

In the sample arm, a sample arm polarization control unit 203 can be used to adjust light polarization state. The light from the fiber coupler 202 can pass through the polarization controller 203 to be collimated by a sample arm collimating lens 204 and reflected by two axial galvanometer mirror scanners (205, 209). Lens 206 can relay the probe beam reflected by the galvanometer mirror scanners (205, 209) into a sample 208. Light from fiber coupler 202 can also pass through a reference arm polarization controller 286 to be collimated by a reference arm collimating lens 213. Lens 287 can focus the beam onto a reference mirror 288 and the light reflected back from mirror can enter the collimator 213.

Via circulators 280 and 285, light scattered back from the sample and reflected back from the reference arm can interfere at fiber coupler 281 to be detected by a balanced detector 282 (e.g., a balanced receiver manufactured by Thorlabs, Inc, Newton, N.J., USA). The signals detected by detector 282 can be sampled by an analog digital conversion unit (e.g., a high speed digitizer manufactured by Innovative Integration, Inc.) and transferred into a computer or other processor for processing.

FIG. 3 schematically illustrates an example broad-spectrum spectral Fourier-domain OCT system 300 for collecting OCT image information. Spectral OCT system 300 comprises a broadband light source 301. Light from source 301 can be coupled into a two by two fiber coupler 302 through a single mode optical fiber. One portion of the light (e.g., 70%) can proceed to the sample arm and the other portion of the light (e.g., 30%) can proceed to the reference arm.

In the sample arm, a sample arm polarization control unit 303 can be used to adjust light polarization state. Light from the fiber coupler 302 can pass through polarization controller 303 to be collimated by sample arm collimating lens 304 and reflected by two axial galvanometer mirror scanners (305, 309). Lens 306 can relay the probe beam reflected by the galvanometer mirror scanners (305, 309) into a sample 308. Light from fiber coupler 302 can also pass through a reference arm polarization controller 386 to be collimated by reference arm collimating lens 313. Lens 387 can focus the beam into a reference mirror 388 and reflect the light back into the collimator.

In this example, light from sample and reference arm can interfere at fiber coupler 302 and collimated by collimating lens 391. The collimated light can pass through grating 392 to generate a spectral signal which can be relayed via lens 393 to a line scan camera 394 for detection. The signals detected by camera 394 can be sampled by an analog digital conversion unit and transferred into a computer or other processor for processing.

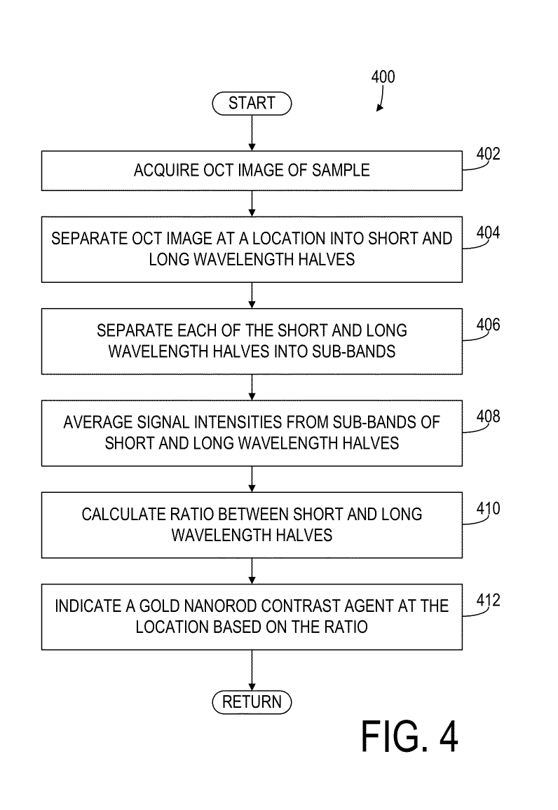

FIG. 4 shows an example method 400 for detecting GNR contrast agents in accordance with various embodiments. Method 400 may be implemented by a system, such as system 100 described above, that includes an OCT system and one or more processors or computing systems, such as computing device 1300 described below. For example, one or more acts described herein may be implemented by one or more processors having physical circuitry programed to perform the acts. In embodiments, one or more steps of method 400 may be automatically performed by one or more processors or computing devices. Further, various acts illustrated in FIG. 4 may be performed in the sequence illustrated, in other sequences, in parallel, or in some cases omitted. Method 400 may be used for detecting a GNR contrast agent at a location, e.g., a pixel location, in an OCT image of a sample acquired by a suitable Fourier-domain OCT system, e.g., a spectral Fourier-domain OCT system or a swept-source (SS) Fourier-domain OCT system.

Method 400 may be used to detect the presence or absence of specifically tuned GNRs within a sample. The GNRs may be engineered for labeling cells with molecular specificity. For example, the GNRs may be engineered to target molecular or cellular structures within a sample, e.g., ligands, antibodies, nanobodies, aptamers, and various other peptides. Thus, in some embodiments the sample may include gold nanorods conjugated with peptides. Such peptides may comprise cell internalizing peptide ligands as demonstrated using a Tat peptide in the example described below. Such an approach may be applicable to similar peptides such as penetratin, transportan, chariot, and maurocalcine, for example.

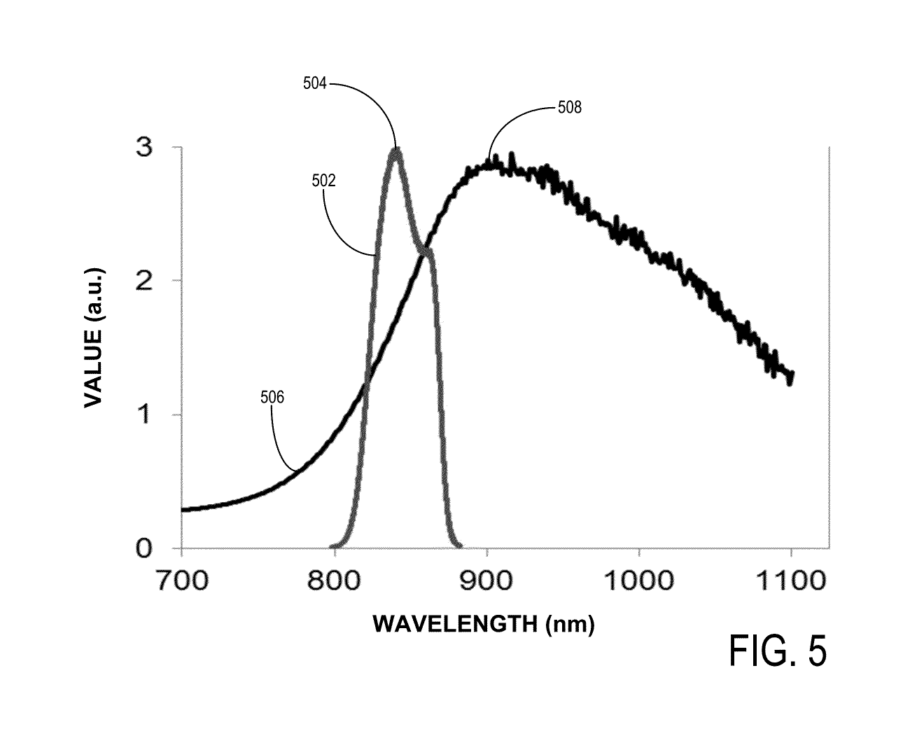

At 402, method 400 includes acquiring an OCT image of a sample. The sample may include GNRs specifically engineered to have a unique spectrally-encoded backscatter/reflectance by tuning their SPR peak to a wavelength that is shifted to one side of the OCT spectral band. As one example, the OCT system may comprise a spectral Fourier-domain OCT system, e.g., as shown in FIG. 3, and the GNR contrast agent may have a surface plasmon resonance at a wavelength greater than the center wavelength of the OCT system. For example, the wavelength of the SPR peak of the gold nanorod contrast agent may be within an approximate range of 700-1400 nm achieved by engineering gold nanorods having diameters within an approximate range of 10-100 nm and lengths within an approximate range of 25-400 nm. In an exemplary embodiment, the wavelength of the SPR peak of the gold nanorod contrast agent may be approximately 900 nm and the center wavelength of the OCT system may be approximately 840 nm. This example is illustrated in FIG. 5. In particular, FIG. 5 shows an example normalized extinction spectra (506) of a GNR with an SPR peak (508) at 900 nm and a normalized intensity spectra (502) of a spectral OCT system having a center wavelength (504) of 840 nm. For example, the GNR contrast agent may comprise gold nanorods having diameters of approximately 10 nm and lengths of approximately 50 nm.

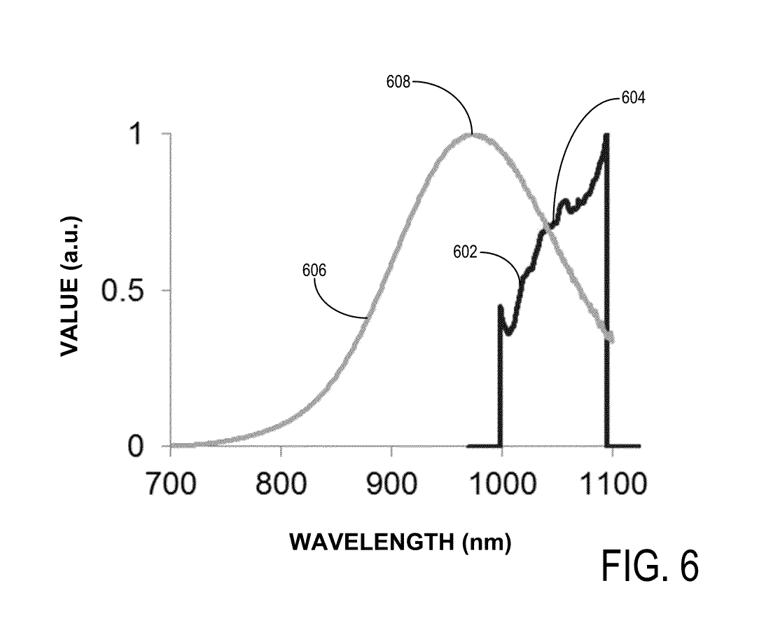

As another example, the OCT system may comprise a swept-source Fourier-domain OCT system, e.g., as shown in FIG. 2, and the GNR contrast agent may have a surface plasmon resonance at a wavelength less than the center wavelength of the OCT system. For example, the wavelength of the SPR peak of the gold nanorod contrast agent may be within an approximate range of 700-1400 nm achieved by engineering gold nanorods having diameters within an approximate range of 10-100 nm and lengths within an approximate range of 25-400 nm. In an exemplary embodiment, the wavelength of the SPR peak of the gold nanorod contrast agent may be approximately 980 nm and the center wavelength of the OCT system may be approximately 1050 nm. This example is illustrated in FIG. 6. In particular, FIG. 6 shows an example normalized extinction spectra (606) of a GNR with an SPR peak (608) at 980 nm and a normalized intensity spectra (602) of a swept-source OCT system having a center wavelength (604) of 1050 nm. For example, the GNR contrast agent may comprise gold nanorods having diameters of approximately 10 nm and lengths of approximately 59 nm.

In embodiments, the OCT data may be received by a computing device from an OCT scanning system via a network or from a storage medium coupled to or in communication with the computing device. The OCT data may be obtained from any suitable Fourier-domain OCT scanning device, e.g., a swept-source OCT scanner or a spectral OCT scanner. Various processing algorithms may be applied to the OCT data in order to condition the image data for parameter extraction. For example, an OCT signal may be derived from an interferogram between a reference light and backscattered/reflected light from the sample and a DC part of the OCT signal may be filtered.

The OCT image may be processed using a spectral fractionation OCT processing technique described in steps 404-410 of method 400 and in the example given below. In particular, at 404, method 400 includes separating the OCT image at a location, e.g., a pixel location, into short and long wavelength halves around a center wavelength of the OCT system. For example, the raw interferogram from any single position in the OCT image may be separated into short and long wavelength halves around the center wavelength of OCT system.

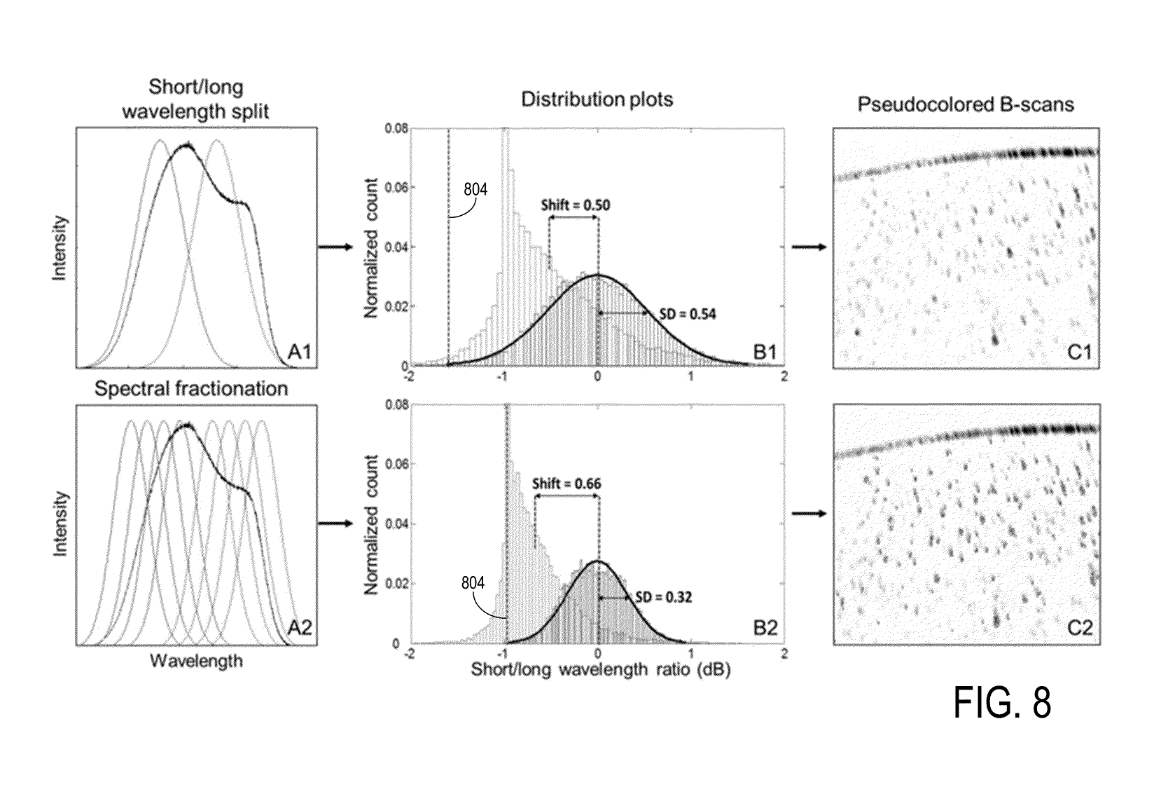

At 406, method 400 may include performing spectral fractionation by separating each of the short and long wavelength halves into sub-bands. For example, a window function may be applied to the OCT image at the location to separate each of the short and long wavelength halves into sub-bands as described in the example given below. Specifically, spectral fractionation may be performing by utilizing Equation 1, described below. As described in the Example below (and illustrated in FIG. 8), performing spectral fractionation suppresses noise by averaging out random spectral shifts caused by speckle (interference between nearby scatterers in tissue) and increases spectral contrast thereby enhancing the ability to identify the presence of GNR within the sample.

At 408, method 400 may include averaging signal intensities from the sub-bands of the short and long wavelength halves. For example, signal intensities from the sub-bands of the short wavelength half may be averaged to obtain a short wavelength OCT depth profile, and signal intensities from the sub-bands of the long wavelength half may be averaged to obtain a long wavelength OCT depth profile. In some examples, the OCT signal from each sub-band may be Fourier-transformed to obtain A-scans. This processing may be performed for all A-scans in consecutive B-frame images acquired at the same location and the sets of results may be averaged.

At 410, method 400 includes calculating a ratio between the short and long wavelength halves. The ratio between the short and long wavelength halves, referred to herein as the “SLoW” ratio, may be calculated for each pixel with signal strength above an intensity threshold. For example, the intensity threshold may comprise the mean signal intensity plus three times the standard deviation (SD) of the signal at a noise region above the sample of interest. In some examples, the ratio between the short and long wavelength halves may be calculated based on the sub-bands of the short and long wavelength halves, e.g., the ratio may be calculated based on the short wavelength OCT depth profile and the long wavelength OCT depth profile. Additionally, in some examples, repeated B-scans at the location may be used in the acquisition of the OCT image and the ratio may be calculated based on OCT image data from the repeated B-scans at the location.

Specifically, the ratio, SLoW (z), between the short and long wavelength halves may calculated according to the following Equation 1:

$$SLoW ( z ) = \frac{{\sum }_{j = 1}^{M} {\sum }_{si = 1}^{N} \vert {I}_{j , si} ( z ) \vert}{{\sum }_{j = 1}^{M} {\sum }_{li = 1}^{N} \vert {I}_{j , li} ( z ) \vert} = \frac{{\sum }_{j = 1}^{M} ( {\sum }_{si = 1}^{N} \vert {\int }_{{k}_{min}}^{{k}_{max}} 2 S ( k ) {r}_{j , s} ( k , z ) {G}_{si} ( k ) cos ( k \cdot z ) d k \vert )}{{\sum }_{j = 1}^{M} ( {\sum }_{li = 1}^{N} \vert {\int }_{{k}_{min}}^{{k}_{max}} 2 S ( k ) {r}_{j , s} ( k , z ) {G}_{si} ( k ) cos ( k \cdot z ) d k \vert )} \qquad ( 1 )$$

In Equation 1, M is a number of repeated B-scans, N is the number of sub-bands for the short/long wavelength halves, I_{j,si}(z) is the OCT signal for the ith sub-band in the short wavelength half at depth z, I_{j,li}(z) is the OCT signal for the ith sub-band in the long wavelength half at depth z, kₘᵢₙ is a minimum wave number of the OCT light source, kₘₐₓ is a maximum wave number of the OCT light source, r_{j,s}(k,z) is a spectral amplitude reflectivity of the sample backscattered/reflected light at depth z for the jth B-scan, Gₛᵢ(k) is a window function used for the ith sub-band in the short band, and Gₗᵢ(k) is a window function used for the ith sub-band in the long band. The SLoW ratio may be generated on a decibel (dB) and used to identify the potential presence or absence of GNRs in the sample as described below. It should be understood by one of ordinary skill in the art that wavenumber and wavelength, as used herein, are equivalent ways of specifying spectral properties of light. In particular, wavelength is inversely related to wavenumber.

At 412, method 400 includes indicating a gold nanorod contrast agent at the location based on the ratio. For example, a presence or absence of gold nanorod contrast agent at the location may be indicated based on the calculated ratio. As one example, when the gold nanorod contrast agent has an SPR peak at a wavelength greater than the center wavelength of the OCT system, indicating a gold nanorod contrast agent at the location based on the ratio may comprise indicating the gold nanorod contrast agent at the location in response to the ratio on a decibel scale less than zero by a predetermined amount. The predetermined amount may be based on a standard deviation of a distribution of ratios of short and long wavelength halves acquired from an OCT image of a sample without a gold nanorod contrast agent. In this example, an absence of a GNR contrast agent at the location may be indicated in response to the ratio on a decibel scale greater than zero by a predetermined amount.

As another example, when the gold nanorod contrast agent has an SPR peak at a wavelength less than the center wavelength of the OCT system, indicating a gold nanorod contrast agent at the location based on the ratio may comprise indicating the gold nanorod contrast agent at the location in response to the ratio on a decibel scale greater than zero by a predetermined amount. As above, the predetermined amount may be based on a standard deviation of a distribution of ratios of short and long wavelength halves acquired from an OCT image of a sample without a gold nanorod contrast agent. In this example, an absence of a GNR contrast agent at the location may be indicated in response to the ratio on a decibel scale less than zero by a predetermined amount.

Indications of the presence of absence of GNRs in the sample may be output by the system in a variety of ways. For example, a visual indication may be output to a display device coupled to the computing device, an audio indication may be output to one or more speakers coupled to the computing device, and/or indication data may be stored in a storage medium of the computing device and/or output to an external device via a network.

### EXAMPLE

The example discussed below illustrates systems and methods for detecting a gold nanorod contrast agent at a location in an OCT image of a sample in accordance with various embodiments. Embodiments may vary as to the methods of obtaining OCT image data, performing OCT data processing, and extracting parameters from the OCT data. The example discussed below is for illustrative purposes and is not intended to be limiting.

This example demonstrates a Fourier-domain optical coherence tomography contrast mechanism using gold nanorod contrast agents and a spectral fractionation processing technique in accordance with various embodiments. The spectral fractionation methodology described herein is used to detect the spectral shift of the backscattered light from the nanorods by comparing the ratio between the short and long wavelength halves of the optical coherence tomography signal intensity. Spectral fractionation further divides the halves into sub-bands to increase spectral contrast. This example demonstrates that this technique can detect gold nanorods in intralipid tissue phantoms. Furthermore, this example demonstrates cellular labeling by gold nanorods using retinal pigment epithelial cells in vitro Embodiments described herein may potentially be applied to in vivo applications.

In this example, imaging experiments were conducted on a commercial Fourier-domain OCT system (RTVue-XR, Optovue, Fremont, Calif.) or a custom-built swept-source OCT system (SSOCT). The commercial system had a center wavelength of ˜840 nm with a bandwidth measured at the full-width-half-maximum of 45 nm and operating speeds of 70 kHz. The system was customized to allow saving of the raw spectral data. A 30 mm lens was used for focusing. The SSOCT system had a center wavelength of 1050 nm with a bandwidth of ˜100 nm and operating speeds of 100 kHz. The SSOCT system had an axial resolution of 7.1 μm in air and a lateral resolution of 19 μm.

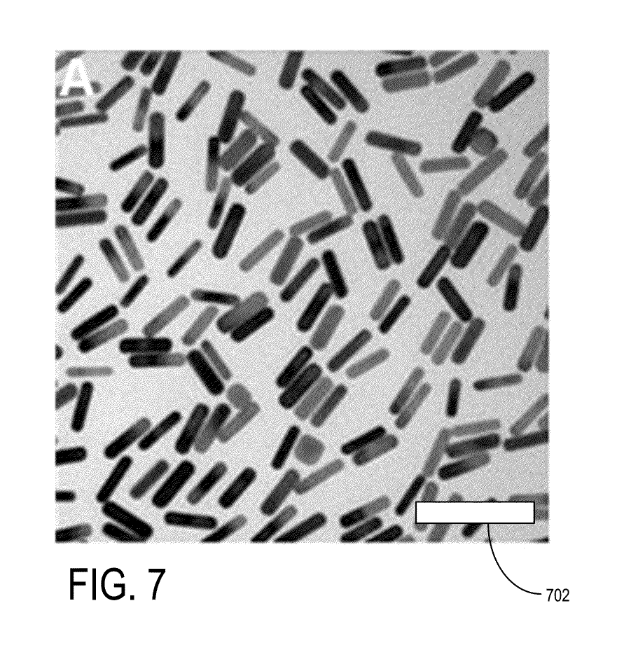

Cetyl trimethylammonium bromide (CTAB) coated gold nanorods were synthesized according to procedures described in Jana et al., Advanced Materials 13, 1389-1393 (2001), which is hereby incorporated by reference. Gold nanorods feature characteristic SPR that is tunable by their aspect ratio. The SPR of 10 nm diameter sized gold nanorods were adjusted to peak values of 900 nm and 980 nm by tuning their length dimensions to 50 nm and 59 nm, respectively. Nanorods were then coated with 1000 molecular weight polyethylene glycol (PEG) via incubation of nanorods with 10-fold molar excess of thiol-PEG-Tat peptide (Laysan Bio, Inc., Arab, Ala.) in phosphate buffered saline (PBS) with pH=7.4 for 12 hours with gentle mixing on a tube rotator. The Tat peptide featured D-amino acids and had the amino acid sequence RKKRRORRR as described in Barnett et al., Invest Ophthalmol Vis Sci 47, 2589-2595 (2006), which is hereby incorporated by reference. Excess PEG-Tat was removed by 6 rounds of centrifugation at 21,000X G with rinsing using PBS. Displacement of CTAB with PEG residues was monitored using zeta potential analysis and surface assisted laser desorption ionization mass spectrometry as described for gold nanorod characterization in Nakamura et al., Nanoscale 3, 3793-3798 (2011), which is hereby incorporated by reference. Furthermore, post-conjugation of PEG-Tat nanorods were characterized for preservation of size using transmission electron microscopy. For example, FIG. 7 shows a transmission electron micrograph of monodisperse GNR coated with polyethylene glycol (PEG) and Tat cell internalization peptides. The scale bar 702 shown in FIG. 7 is 100 nm.

Tissue phantoms (5 mL) of intralipid, GNR, and GNR-in-intralipid were all prepared by serial dilution from stock solutions and imaged in 10 mL test tubes using OCT. Intralipid 20% stock solution (Intralipid 20%, emulsion, Sigma Aldrich) was diluted down to 0.1% using molecular grade water. GNR stock solution (5×10¹¹ nanorods/ml) was serially diluted using molecular grade water to 1.5×10¹⁹ nanorod/mL. The GNR-in-intralipid solution was prepared by first diluting the stock intralipid solution to 0.1%. This solution was then used to dilute stock GNR solution, resulting in a final 0.1% intralipid sample with 1.5×10¹⁹ nanorod/mL.

Gelatin-coated plates with no cells, unlabeled retinal pigment epithelial (RPE) cells, and GNR-labeled RPE cells were imaged in vitro using OCT. Cell plates containing approximately 500,000 RPE stem cells were incubated with 1×10⁹ Tat-coated GNR for 4 hours at 37° C. on an agitator (at 30 RPM). Afterwards, the cell plates were washed free of GNR by triplicate rinsing with warmed Hanks' balanced salt solution (HBSS), thus allowing for only GNR taken up by RPE cells to remain. Cells were then trypsinized, centrifuged and fixed using 1 mL 10% neutral buffered formalin at room temp for 10 min.

Prior to the addition of labeled cells, a basal layer was first prepared in the wells that would contain no cells, labeled cells, or unlabeled cells using 3 mL of 1% gelatin. This created a depth of approximately 300 μm above the plastic bottom of the well plate for imaging purposes. Cells were then resuspended in an additional 2 mL of 1% gelatin. This was subsequently added to the previously solidified, gelatin coated plates. This provided an additional depth of approximately 200 μm for imaging.

The OCT signal was derived from the interferogram between a reference light and backscattered/reflected light from the sample. After filtering the DC part, the interferogram signal can be expressed as the following Equation 2:

l(z)=∫2S(k)rᵣ(k)rₛ(k,z)cos(k·z)dk (2)

In Equation 2, S(k) is the power spectrum of the OCT light source, rᵣ(k) is the spectral amplitude reflectivity of the reference mirror, rₛ(k, z) is the spectral amplitude reflectivity of the sample backscattered/reflected light at depth z, k is the wavenumber, and z is the path difference between sample and reference mirror. For a typical OCT setup, rᵣ(k) is constant and wavelength independent because a mirror is usually used in the reference arm. Biological tissues can have wavelength dependent absorption and scattering properties, and therefore, the OCT sample arm reflectance rₛ(k, z) is generally wavelength dependent. However, the wavelength dependence of biological tissue is typically weak for near-infrared wavelengths, and there is no large systematic spectral shift as with GNR contrast agents. A spectral shift was detected using the ratio between the short and long wavelength halves (SLoW ratio) of the OCT signal amplitude. In order to improve the signal-to-noise ratio, the short/long band may be further split into several sub-bands through a window function and repeated B-scans can be used to reduce speckle noise. Through this spectral fractionation, the modified SLoW ratio was calculated according to Equation 1 above.

Based on the measured light source spectrum of the Fourier-domain OCT system (RTVue-XR) and measured extinction spectrum of the synthesized GNR with an SPR peak at 900 nm, the normalized SLoW ratio was numerically simulated and a value of −0.683 decibels (dB) was found for 4 sub-bands in the short/long band. Normalization was performed to take the light source spectrum into consideration so that a SLoW ratio of zero dB would be found for wavelength independent samples.

The collected OCT data were analyzed with the spectral fractionation OCT processing technique. The spectral fractionation OCT processing technique is illustrated in FIG. 8. The raw interferogram from any single position was first separated into short and long wavelength halves around the center wavelength of the OCT system as shown in FIG. 8, A1. In particular, FIG. 8, A1 shows a raw interferogram (black) split into short (blue) and long (red) wavelength halves. Each half was then further spectrally fractionated into four narrower sub-bands using Gaussian filters to improve detection (FIG. 8, A2); similar to what was described in Jia et al., Opt Express 20, 4710-4725 (2012), which is hereby incorporated by reference. The OCT signal from each sub-band was then Fourier-transformed to obtain A-scans as in conventional Fourier-domain OCT algorithms. The resulting signal intensities from the spectral bands were averaged together to obtain short and long wavelength OCT depth profiles. This processing was done for all A-scans in 10 consecutive B-frame images acquired at the same location. The 10 sets of results were averaged.

To evaluate the spectral shift of the OCT signal as a result of the GNR, the SLoW intensity ratio (Equation 1) was calculated for each pixel with signal strength above an intensity threshold (FIGS. 8, B1 and B2). The intensity threshold used in this example was defined as the mean plus three times the standard deviation (SD) of the signal at a noise region above the sample of interest. The multi-frame and multi-band imaging/processing steps were taken to suppress speckle noise and decrease the spread of the SLoW ratio in tissue. When GNR with SPR peaks at 900 nm (FIG. 5) were imaged with the 840 nm commercial OCT system, the longer wavelength bands had stronger backscattered/reflected signals. Thus, when the SLoW ratio is presented on a dB scale, a negative value indicated the potential presence of GNR. Conversely, when GNR with SPR peaks at 980 nm (FIG. 6) were imaged with the 1050 nm SSOCT system, the shorter wavelength bands had stronger backscattered/reflected signals. Thus, a positive value of the SLoW ratio presented on a dB scale indicated the potential presence of GNR.

As a first step, tissue phantoms were used to test the detection methodology described herein. B-scan images of 0.1% intralipid, GNR with SPR peaks at 900 nm, and GNR mixed with intralipid were taken on the 840 nm commercial OCT system. The images were processed using the spectral fractionation technique described above, and the SLoW ratios were calculated. The OCT images were also processed without splitting the short/long wavelength bands into 4 sub-bands to show the effect of that step (FIG. 8). Histogram distribution plots of the SLoW ratios on a dB scale for the intralipid and GNR samples were generated (FIGS. 8, B1 and B2). The histograms were normalized to the total number of pixels in the B-scan which met the initial intensity threshold. The intralipid showed a normal distribution centered on zero (FIGS. 8, B1 and B2). Without the spectral fractionation sub-band split, the SD of the intralipid distribution was greater, 0.54 versus 0.32.

A SLoW ratio shift was then defined between the distribution plots of the two samples as the difference between the means (FIGS. 8, B1 and B2). Without the spectral fractionation sub-band split, the mean from the GNR sample was within one SD of the intralipid distribution. Using a cutoff of 3.09 SD or −1.67 dB (lines 804 in FIGS. 8, B1 and B2) as the criteria of identifying the presence of GNR, 1% of the SLoW signal from the GNR sample remained. With spectral fractionation, the mean from the GNR sample showed a SLoW ratio with a mean ˜2 SD less than that of the intralipid distribution. Using a cutoff of 3.09 SD or −1.0 dB SLoW ratio as the cutoff (line 804 in FIG. 8), the GNR signal could be cleanly separated from the intralipid signal. Specifically, 18% of the SLoW signal from the GNR sample was less than −1 dB compared to only 0.1% from the intralipid sample. The difference when analyzing with spectral fractionation was clearly seen when the SLoW ratios were pseudocolored on a dB scale for locations having a SLoW ratio less than the mean from the intralipid sample minus 3.09 SD (red) and greater than the mean plus 3.09 SD (blue) onto the B-scan images from the GNR sample (FIG. 8C). To simplify the terminology, the term spectral contrast (S_{C}) was introduced and defined using the following Equation 3:

$${S}_{C} = \frac{{M}_{GNR} - {M}_{S}}{{δ}_{S}} \qquad ( 3 )$$

In Equation 3, M_{GNR} is the mean SLoW ratio on a dB scale from the GNR sample distribution plot, M_{S} is the mean from the GNR free sample, and δ_{S} the standard deviation from the GNR free sample. M_{GNR}−M_{S} represents the SLoW ratio shift. A greater spectral contrast value would indicate an enhanced ability to identify the presence of GNR within the mixture. Without spectral fractionation, the spectral contrast of 0.1% intralipid and GNR with SPR peaks at 900 nm was then 0.93. With spectral fractionation, the spectral contrast increased to 2.06.

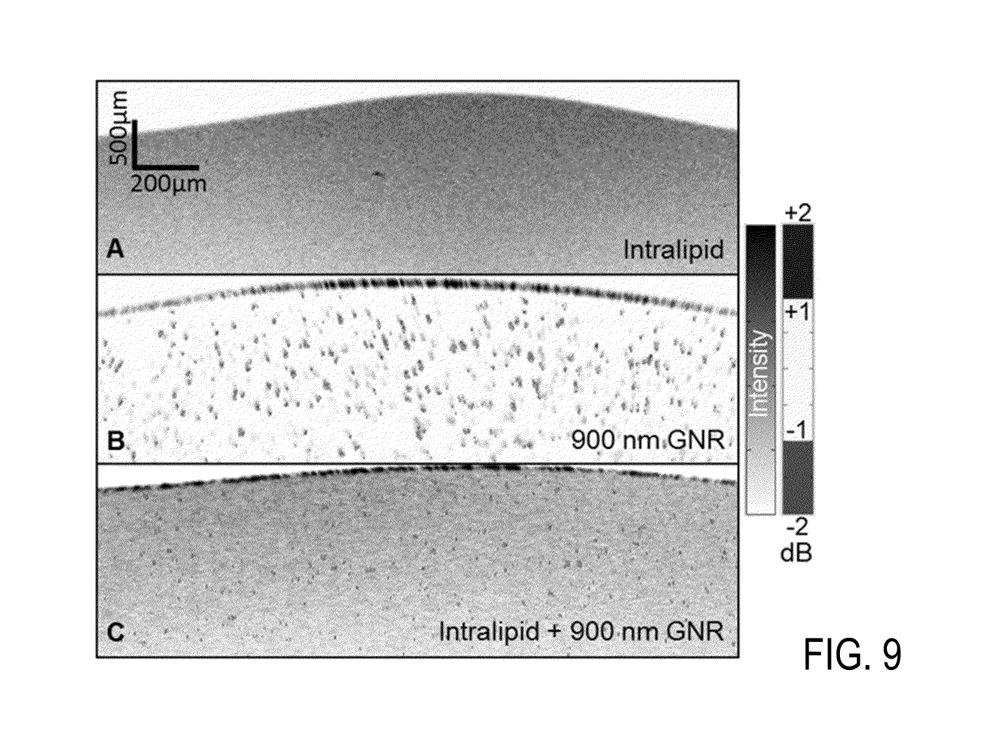

Using the −1 and +1 dB cutoffs, the SLoW ratio information was pseudocolored over the B-scan images from the intralipid, GNR with SPR peaks at 900 nm, and GNR mixed with intralipid samples. As was done previously, blue was used to show the regions with SLoW ratios greater than 1 dB, and red was used to show the regions with SLoW ratios less than −1 dB. Due to speckle noise, the intralipid (FIG. 9A) showed a few colored pixels. The red GNR signal could be clearly visualized by itself (FIG. 9B) and when mixed with intralipid (FIG. 9C). The high intensity edge at the top of FIGS. 9B and 9C was from the test tube surface and was excluded from the analysis. In FIG. 9, the OCT signal intensity is shown on an inverse gray scale and the SLoW ratio is color-coded with cutoffs set at ±3.09 SD (±1 dB) of the spectral distribution of intralipid (FIG. 8, B2). Intralipid (FIG. 9A) showed rare color pixels due to noise. The red GNR signal could be clearly visualized by itself (FIG. 9B) and when mixed with intralipid (FIG. 9C).

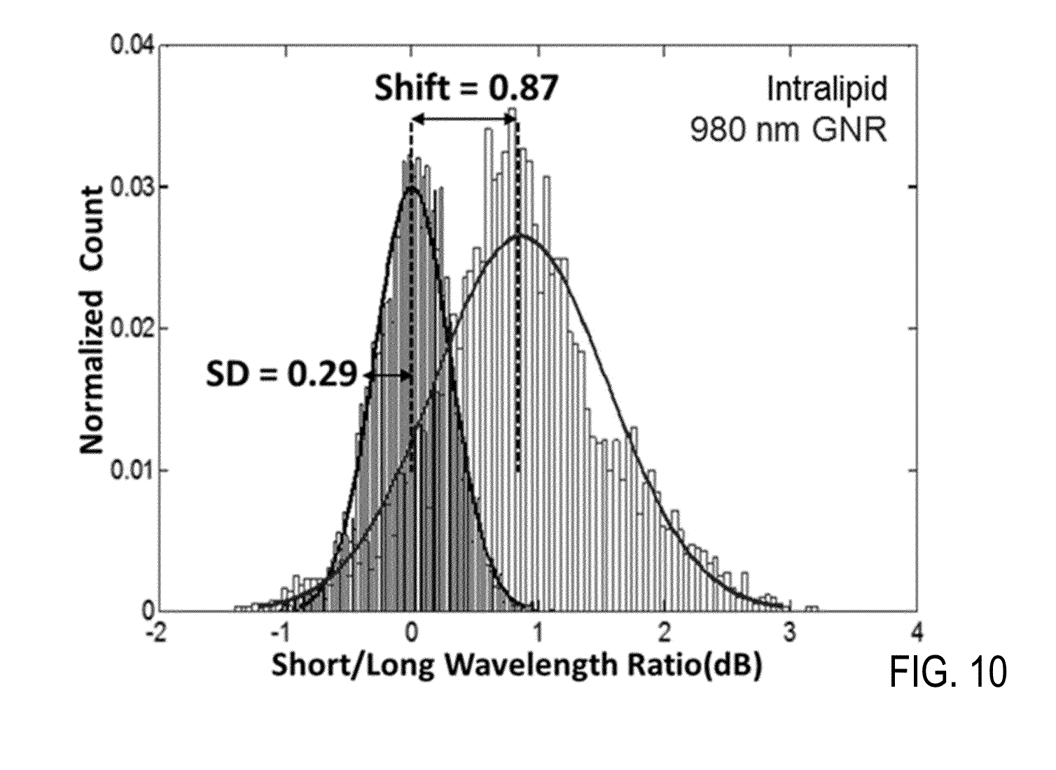

To show detection of GNR with positive SLoW ratios on a dB scale, B-scan images of 0.1% intralipid and GNR with SPR peaks at 980 nm were taken on the 1050 nm SSOCT system. SLoW ratios were calculated as before (Equation 1), and after conversion to a dB scale, histogram distribution plots of the SLoW ratios for the intralipid and GNR samples were generated (FIG. 10). The histograms were normalized to the total number of pixels in the B-scan which met the initial intensity threshold. Again, the intralipid showed a normal distribution centered on zero. The GNR, on the other hand, showed a SLoW ratio with a mean 3 SD more than that of the intralipid distribution. With a 3.09 SD or 0.9 dB SLoW ratio as the cutoff, the GNR signal could be cleanly separated from the intralipid signal. The spectral contrast for this sample, GNR, and OCT system combination was 3.

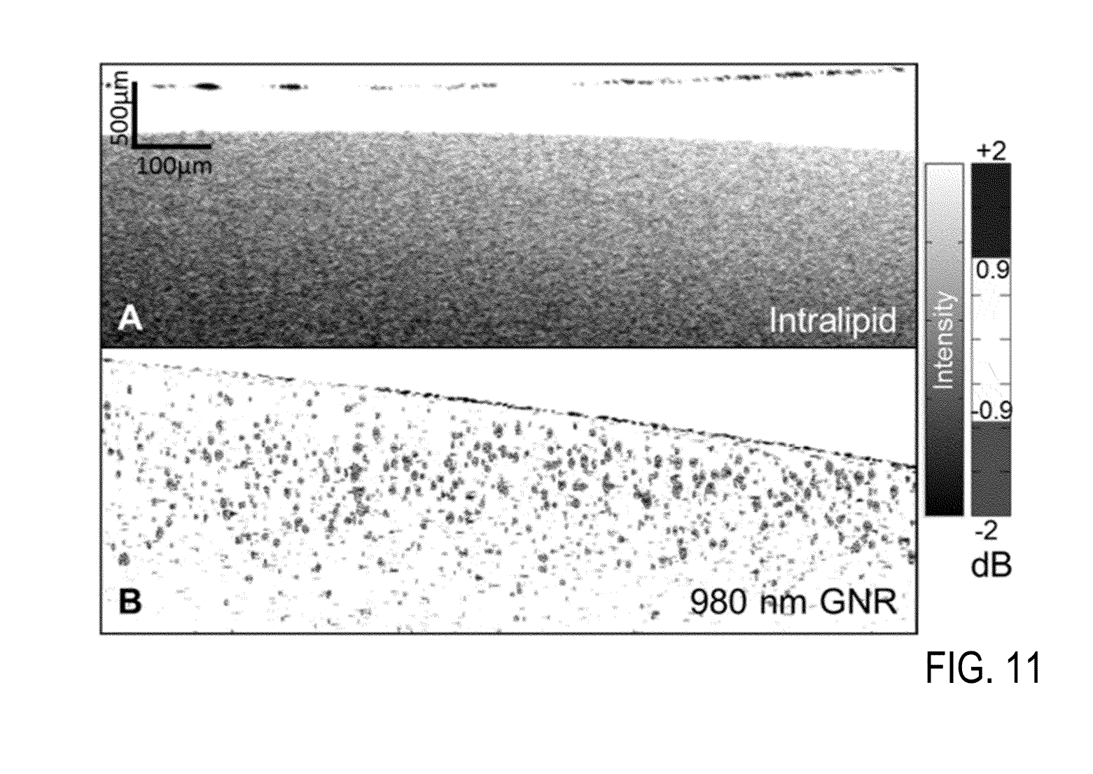

Using −0.9 and +0.9 dB as cutoffs, the SLoW ratio information was again pseudocolored over the B-scan images. Blue was used to show the regions with SLoW ratios greater than 0.9 dB, and red was used to show the regions with SLoW ratios less than −0.9 dB. Due to speckle noise, the intralipid (FIG. 11A) showed a few colored pixels. The blue GNR signal could be clearly visualized (FIG. 11B). Specifically, 43% of the SLoW signal from the GNR sample was greater than 0.9 dB compared to only 0.1% from the intralipid sample. In FIG. 11, the OCT signal intensity is shown on an inverse gray scale, and SLoW ratio information is color-coded with cutoffs set at ±3.09 SD (±0.9 dB) of the spectral distribution of intralipid (FIG. 10). Intralipid (FIG. 11A) showed sparse color pixels due to noise. The blue GNR signal (FIG. 11B) could be clearly visualized.

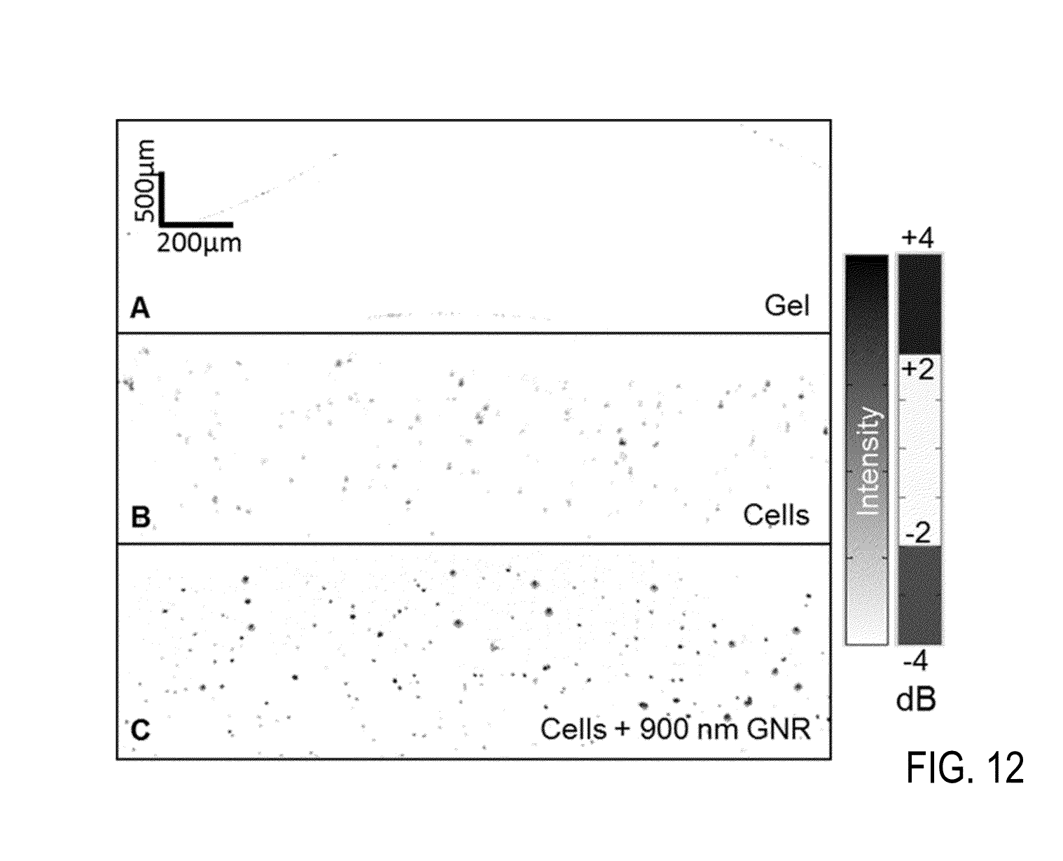

The detection methodology was then tested on cultured RPE cells. B-scan images of 1% gelatin, unlabeled RPE cells, and RPE cells labeled with SPR 900 nm GNR coated with PEG and Tat were taken. Based on the SD of the SLoW histogram from unlabeled RPE cells, new cutoffs of −2 and +2 dB were used in this experiment. Using this criterion, 5% of the SLoW signal from the GNR-labeled RPE cells was less than −2 dB. GNR-labeled RPE cells (FIG. 12C) could be distinguished (red dots), in sharp contrast with unlabeled cells (FIG. 12B). A few GNR-labeled cells unexpectedly showed blue, perhaps due to heterogeneity in GNR dimensions and SPR or the interaction of multiple GNR closely localized in the same cell. In FIG. 12, the spectral shift of the backscattered/reflected OCT signal is shown as a SLoW ratio with a blue-white-red color scheme with cutoffs set at ±3.09 SD (±2 dB) of the spectral distribution of unlabeled cells. The intensity of the OCT signal is shown on an inverse gray scale. GNR-labeled RPE cells (FIG. 12C) could be distinguished by their spectral shift (red dots), in sharp contrast with unlabeled cells (FIG. 12B). A few GNR-labeled cells showed blue, most likely due to the spread in the GNR spectrum associated with GNR heterogeneity or aggregation.

This example demonstrated the detection of GNR with SPR tuned to 900 nm in tissue phantoms and RPE cells with an 840 nm commercial OCT system. Additionally, the detection of GNR with SPR tuned to 980 nm with a 1050 nm SSOCT system was shown. The formulated GNRs showed stronger backscattered/reflected signals at shorter or longer wavelengths and were thus identifiable when comparing the SLoW ratio after spectral fractionation analysis. This example demonstrated that GNR gave rise to distinct negative or positive SLoW ratios on a dB scale, which was then used to distinguish GNR from their surrounding environment. The term spectral contrast was defined which can be used to conceptualize and assess the effectiveness of any given GNR, sample, and OCT system combination.

In this example, not all GNR signal gave rise to a SLoW ratio beyond the cutoffs, which may have been partially due to the purity of the GNR sample. In support of this was the presence of cells in the in vitro experiments with high positive SLoW ratios; this suggested that the GNR may not have been homogenously dispersed. Additionally, some false signals were observed in the intralipid samples. In order to address these issues, a wider spectrum light source may be utilized and the size and aspect ratio of GNR particles may be tuned to increase the spectral contrast. In particular, the slope of the GNR extinction curve, which has the same shape as the reflectance/scattering curve within the OCT spectral window is a determining factor of spectral contrast (see He et al., The Journal of Physical Chemistry C 114, 2853-2860 (2010) and Qiu et al., Biomed Opt Express 1, 135-142 (2010), both of which are hereby incorporated by reference). Based on the simulations performed in this example, capturing more of the slope within a wider OCT spectral window and a steeper slope both lead to increased contrast. As an example of the former, increasing the bandwidth of the 840 nm OCT system by ˜40% can increase the spectral contrast between the GNR with SPR at 900 nm and intralipid by ˜30%. The aforementioned slope can be increased by narrowing the SPR bandwidth. In general, the SPR spectrum is narrower and reflectance/scatter greater with GNR with lower aspect ratios. In addition, having a more homogeneous distribution of GNR size and shape can help to reduce broadening of the SPR spectrum. On the other hand, GNR with greater aspect ratios shift their SPR peak to longer wavelengths. Therefore, optimal performance of the approaches described herein may depend on engineering GNR to have an optimal balance of high reflectance and a narrow SPR spectrum with a peak located to one side of the OCT spectrum. Equation 1 described above, provides a framework which can be used to assess these relationships between the GNR extinction spectrum, OCT spectrum, and spectral contrast.

In some embodiments, the above described methods and processes may be tied to a computing system, including one or more computers. In particular, the methods and processes described herein, e.g., method 400 described above, may be implemented as a computer application, computer service, computer API, computer library, and/or other computer program product.

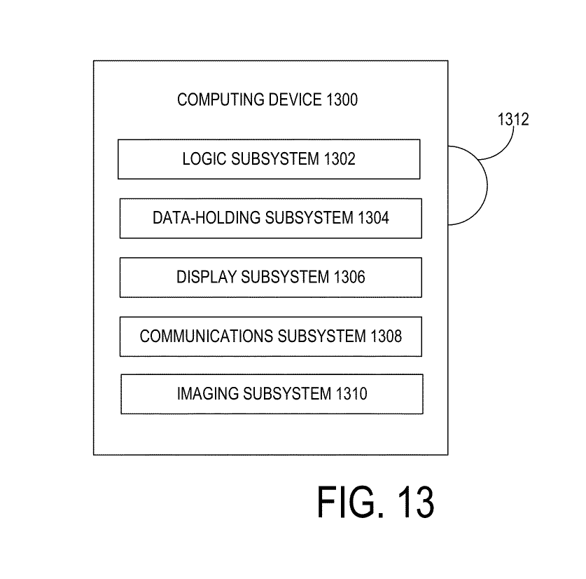

FIG. 13 schematically shows a non-limiting computing device 1300 that may perform one or more of the above described methods and processes. For example, computing device 1300 may represent a processor included in system 100 described above, and may be operatively coupled to, in communication with, or included in an OCT system or OCT image acquisition apparatus. Computing device 1300 is shown in simplified form. It is to be understood that virtually any computer architecture may be used without departing from the scope of this disclosure. In different embodiments, computing device 1300 may take the form of a microcomputer, an integrated computer circuit, microchip, a mainframe computer, server computer, desktop computer, laptop computer, tablet computer, home entertainment computer, network computing device, mobile computing device, mobile communication device, gaming device, etc.

Computing device 1300 includes a logic subsystem 1302 and a data-holding subsystem 1304. Computing device 1300 may optionally include a display subsystem 1306 and a communication subsystem 1308, and/or other components not shown in FIG. 13. Computing device 1300 may also optionally include user input devices such as manually actuated buttons, switches, keyboards, mice, game controllers, cameras, microphones, and/or touch screens, for example.

Logic subsystem 1302 may include one or more physical devices configured to execute one or more machine-readable instructions. For example, the logic subsystem may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. Additionally or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Data-holding subsystem 1304 may include one or more physical, non-transitory, devices configured to hold data and/or instructions executable by the logic subsystem to implement the herein described methods and processes. When such methods and processes are implemented, the state of data-holding subsystem 1304 may be transformed (e.g., to hold different data).

Data-holding subsystem 1304 may include removable media and/or built-in devices. Data-holding subsystem 1304 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1304 may include devices with one or more of the following characteristics: volatile, nonvolatile, dynamic, static, read/write, read-only, random access, sequential access, location addressable, file addressable, and content addressable. In some embodiments, logic subsystem 1302 and data-holding subsystem 1304 may be integrated into one or more common devices, such as an application specific integrated circuit or a system on a chip.

FIG. 13 also shows an aspect of the data-holding subsystem in the form of removable computer-readable storage media 1312, which may be used to store and/or transfer data and/or instructions executable to implement the herein described methods and processes. Removable computer-readable storage media 1312 may take the form of CDs, DVDs, HD-DVDs, Blu-Ray Discs, EEPROMs, flash memory cards, and/or floppy disks, among others.

When included, display subsystem 1306 may be used to present a visual representation of data held by data-holding subsystem 1304. As the herein described methods and processes change the data held by the data-holding subsystem, and thus transform the state of the data-holding subsystem, the state of display subsystem 1306 may likewise be transformed to visually represent changes in the underlying data. Display subsystem 1306 may include one or more display devices utilizing virtually any type of technology. Such display devices may be combined with logic subsystem 1302 and/or data-holding subsystem 1304 in a shared enclosure, or such display devices may be peripheral display devices.

When included, communication subsystem 1308 may be configured to communicatively couple computing device 1300 with one or more other computing devices. Communication subsystem 1308 may include wired and/or wireless communication devices compatible with one or more different communication protocols. As non-limiting examples, the communication subsystem may be configured for communication via a wireless telephone network, a wireless local area network, a wired local area network, a wireless wide area network, a wired wide area network, etc. In some embodiments, the communication subsystem may allow computing device 1300 to send and/or receive messages to and/or from other devices via a network such as the Internet.

When included, imaging subsystem 1310 may be used acquire and/or process any suitable image data from various sensors or imaging devices in communication with computing device 1300. For example, imaging subsystem 1310 may be configured to acquire OCT image data as part of an OCT system, e.g., OCT system 102 described above. Imaging subsystem 1310 may be combined with logic subsystem 1302 and/or data-holding subsystem 1304 in a shared enclosure, or such imaging subsystems may comprise periphery imaging devices. Data received from the imaging subsystem may be held by data-holding subsystem 1304.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A computerized method for detecting a gold nanorod contrast agent at a location in an optical coherence tomography (OCT) image of a sample, comprising:
separating the OCT image at the location into short and long wavelength halves around a center wavelength of the OCT system;
calculating a ratio between the short and long wavelength halves; and
indicating a gold nanorod contrast agent at the location based on the ratio.

2. The method of claim 1, wherein the OCT image of the sample is acquired from an OCT system having only a single light source with a continuous spectrum.

3. The method of claim 1, wherein the center wavelength of the OCT system is greater or less than the wavelength of the SPR peak of the gold nanorod contrast agent.

4. The method of claim 1, further comprising, separating each of the short and long wavelength halves into sub-bands, and wherein the ratio between the short and long wavelength halves is calculated based on the sub-bands of the short and long wavelength halves.

5. The method of claim 4, wherein separating each of the short and long wavelength halves into sub-bands comprises applying a window function to the OCT image at the location.

6. The method of claim 4, further comprising averaging the signal intensities from the sub-bands of the short wavelength half to obtain a short wavelength OCT depth profile, averaging the signal intensities from the sub-bands of the long wavelength half to obtain a long wavelength OCT depth profile, and wherein the ratio is calculated based on the short wavelength OCT depth profile and the long wavelength OCT depth profile.

7. The method of claim 4, wherein separating each of the short and long wavelength halves into sub-bands comprises separating each of the short and long wavelength halves into four sub-bands.

8. The method of claim 1, wherein the ratio is calculated based on OCT image data from repeated B-scans at the location.

9. The method of any of claims 2-8, wherein the ratio, SLoW (z), between the short and long wavelength halves is calculated according to the equation
$$SLoW ( z ) = \frac{{\sum }_{j = 1}^{M} {\sum }_{si = 1}^{N} \vert {I}_{j , si} ( z ) \vert}{{\sum }_{j = 1}^{M} {\sum }_{li = 1}^{N} \vert {I}_{j , li} ( z ) \vert} = \frac{{\sum }_{j = 1}^{M} ( {\sum }_{si = 1}^{N} \vert {\int }_{{k}_{min}}^{{k}_{max}} 2 S ( k ) {r}_{j , s} ( k , z ) {G}_{si} ( k ) cos ( k \cdot z ) d k \vert )}{{\sum }_{j = 1}^{M} ( {\sum }_{li = 1}^{N} \vert {\int }_{{k}_{min}}^{{k}_{max}} 2 S ( k ) {r}_{j , s} ( k , z ) {G}_{si} ( k ) cos ( k \cdot z ) d k \vert )}$$
where M is a number of repeated B-scans, N is the number of sub-bands for the short/long wavelength halves, I_{j,si}(z) is the OCT signal for the ith sub-band in the short wavelength half at depth z, I_{j,li}(z) is the OCT signal for the ith sub-band in the long wavelength half at depth z, kₘᵢₙ is a minimum wave number of the OCT light source, kₘₐₓ is a maximum wave number of the OCT light source, r_{j,s}(k,z) is a spectral amplitude reflectivity of the sample backscattered/reflected light at depth z for the jth B-scan, Gₛᵢ(k) is a window function used for the ith sub-band in the short band, and Gₗᵢ(k) is a window function used for the ith sub-band in the long band.

10. The method of claim 1, wherein the gold nanorod contrast agent has a surface plasmon resonance (SPR) peak at a wavelength greater than the center wavelength of the OCT system, and wherein indicating a gold nanorod contrast agent at the location based on the ratio comprises indicating the gold nanorod contrast agent at the location in response to the ratio on a decibel scale less than zero by a predetermined amount.

11. The method of claim 10, wherein the predetermined amount is based on a standard deviation of a distribution of ratios of short and long wavelength halves acquired from an OCT image of a sample without a gold nanorod contrast agent.

12. The method of claim 10, wherein the wavelength of the SPR peak of the gold nanorod contrast agent is within an approximate range of 700-1400 nm and wherein the center wavelength of the OCT system is less than the wavelength of the SPR peak of the gold nanorod contrast agent.

13. The method of claim 12, wherein the gold nanorod contrast agent comprises gold nanorods having diameters within an approximate range of 10-100 nm and lengths within an approximate range of 25-400 nm.

14. The method of claim 12, wherein the wavelength of the SPR peak of the gold nanorod contrast agent is approximately 900 nm, and wherein the center wavelength of the OCT system is approximately 840 nm.

15. The method of claim 14, wherein the gold nanorod contrast agent comprises gold nanorods having diameters of approximately 10 nm and lengths of approximately 50 nm.

16. The method of claim 10, wherein the OCT system comprises a spectral Fourier-domain OCT system.

17. The method of claim 1, wherein the gold nanorod contrast agent has a surface plasmon resonance (SPR) peak at a wavelength less than the center wavelength of the OCT system, and wherein indicating a gold nanorod contrast agent at the location based on the ratio comprises indicating the gold nanorod contrast agent at the location in response to the ratio on a decibel scale greater than zero by a predetermined amount.

18. The method of claim 17, wherein the predetermined amount is based on a standard deviation of a distribution of ratios of short and long wavelength halves acquired from an OCT image of a sample without a gold nanorod contrast agent.

19. The method of claim 17, wherein the wavelength of the SPR peak of the gold nanorod contrast agent is within an approximate range of 700-1400 nm and wherein the center wavelength of the OCT system is greater than the wavelength of the SPR peak of the gold nanorod contrast agent.

20. The method of claim 19, wherein the gold nanorod contrast agent comprises gold nanorods having diameters within an approximate range of 10-100 nm and lengths within an approximate range of 25-400 nm.

21. The method of claim 19, wherein the wavelength of the SPR peak of the gold nanorod contrast agent is approximately 980 nm and wherein the center wavelength of the OCT system is approximately 1050 nm.

22. The method of claim 21, wherein the gold nanorod contrast agent comprises gold nanorods having diameters of approximately 10 nm and lengths of approximately 59 nm.

23. The method of claim 1, wherein the OCT image is acquired by a Fourier-domain OCT system.

24. The method of claim 24, wherein the Fourier-domain OCT system comprises a spectral OCT system.

25. The method of claim 24, wherein the Fourier-domain OCT system comprises a swept source OCT system.

26. The method of claim 1, wherein the sample includes gold nanorods conjugated with peptides.

27. The method of claim 26, wherein the peptides comprise cell internalizing peptide ligands.

28. A system for detecting a gold nanorod contrast agent at a location in an OCT image of a sample, comprising:
an OCT system configured to acquire an OCT image of a sample, the OCT system having a center wavelength;
a logic subsystem; and
a data holding subsystem comprising machine-readable instructions stored thereon that are executable by the logic subsystem to perform the method of claim 1.

29. The system of claim 28, wherein the center wavelength of the OCT system is greater or less than the wavelength of the SPR peak of the gold nanorod contrast agent.

30. The system of claim 28, wherein the OCT system comprises a Fourier-domain OCT system.

31. The system of claim 30, wherein the OCT system comprises a swept-source OCT system.

32. The system of claim 31, wherein the gold nanorod contrast agent has a surface plasmon resonance (SPR) peak at a wavelength within an approximate range of 700-1400 nm and wherein the center wavelength of the OCT system is greater than the wavelength of the SPR of the gold nanorod contrast agent.

33. The system of claim 32, wherein the gold nanorod contrast agent has a surface plasmon resonance (SPR) peak at a wavelength of approximately 980 nm and wherein the center wavelength of the OCT system is approximately 1050 nm.

34. The system of claim 30, wherein the OCT system comprises a spectral OCT system.

35. The system of claim 34, wherein the gold nanorod contrast agent has a surface plasmon resonance (SPR) peak at a wavelength within an approximate range of 700-1400 nm and wherein the center wavelength of the OCT system is less than the wavelength of the SPR peak of the gold nanorod contrast agent.

36. The system of claim 35, wherein the gold nanorod contrast agent has a surface plasmon resonance (SPR) peak at a wavelength of approximately 900 nm, and wherein the center wavelength of the OCT system is approximately 840 nm.

37. The system of claim 28, wherein the OCT system has only a single light source with a continuous spectrum.
